(19) United States
(12) Patent Application Publication
(10) Pub. No.: US 2017/0176439 A1
(43) Pub. Date: Jun. 22, 2017

(54) MARKERS AND THERAPEUTIC INDICATORS FOR GLIOBLASTOMA MULTIFORME (GBM)

(71) Applicant: Institute for Systems Biology, Seattle, WA (US)

(72) Inventors: Dhimankrishna GHOSH, Seattle, WA (US); Charles S. COBBS, Mercer Island, WA (US); Nathan D. PRICE, Seattle, WA (US); Leroy HOOD, Seattle, WA (US)

(73) Assignee: Institute for Systems Biology, Seattle, WA (US)

(21) Appl. No.: 15/390,276

(22) Filed: Dec. 23, 2016

Related U.S. Application Data
(63) Continuation-in-part of application No. PCT/US2015/038043, filed on Jun. 26, 2015.
(60) Provisional application No. 62/017,748, filed on Jun. 26, 2014.

(51) Int. Cl. G01N 33/574

(52) U.S. Cl. CPC G01N 33/57407; G01N 2800/56

(57) ABSTRACT

A signature of proteins occurring in glioblastoma multiforme (GBM) tissue comprised of 33 cell surface proteins (GBMSig) that distinguish subjects with GBM from healthy controls with more than 98% accuracy is described. In addition, four of the members of this signature are particularly useful as blood-borne markers of GBM. Certain other members of the signature are indicators of the possible efficacy of the use of TGF-β1 inhibitors in the treatment of this condition. Methods to treat and to stratify GBM based on GBMSig proteins are also disclosed.

### CROSS-REFERENCE TO RELATED APPLICATION

This application is a continuation-in-part of PCT/US2015/038043, filed 26 Jun. 2015, which claims priority from U.S. provisional application 62/017,748 filed 26 Jun. 2014. The contents of these documents are incorporated herein by reference.

### STATEMENT OF RIGHTS TO INVENTIONS MADE UNDER FEDERALLY SPONSORED RESEARCH

This work was supported in part by National Institutes of Health/National Cancer Institute (NIH/NCI) NanoSystems Biology Cancer Center grant U54 CA151819A (LH) and an NIH/NCI Howard Temin Pathway to Independence Award in Cancer Research (NDP). The U.S. government has certain rights in this invention.

### TECHNICAL FIELD

The invention relates to the malignant primary brain tumor most common in adults, glioblastoma multiforme (GBM). In particular, it concerns identification of bloodborne markers for this condition as well as markers that indicate the viability of potential therapies.

### BACKGROUND ART

Glioblastoma multiforme (GBM) is the most aggressive and malignant of all adult brain tumors. Tumor cells can infiltrate to local and distant regions of brain via subependymal zones and basement membrane. Such GBM pathology results in the development of intracranial pressure, cognitive dysfunction and associated symptoms that invariably culminate in death.

Relative to significant improvements in treatment and survival outcomes for other cancers over the years, the median survival rate for GBM even under treatment is still only 1.5 years—a figure that has been largely unchanged for decades. In United States alone, 9,500 new GBM cases are registered each year with 13,000 deaths (Jemal, A., et al., Cancer J. Clin. (2010) 60:277-300).

Because of the short survival time of GBM patients, there is a very short therapeutic window in which to test multiple therapies in hopes of finding one that might help the patient. There is a desperate need for effective blood-based diagnostics to open a longer therapeutic window and provide better assessment of the molecular responses to therapies.

In recent years, advancements in omics technologies have enabled generation of sensitive and high throughput analytical data and have begun to delineate the GBM disease-perturbed networks for better insight into disease mechanism and to provide diagnostics. Molecular subclasses of GBM from DNA microarray analyses of astrocytoma samples (N=76) by Phillips, H. S., et al., Cancer Cell (2006) 9:157-173, and subsequent follow-up study by Colman, H., et al., Neuro. Oncol. (2010) 12:49-57, have resulted in a proposed 38-gene survival set and a 9-gene set associated with poor prognosis. More recently, The Cancer Genome Atlas (TCGA) project has provided a multidimensional omics view of the aberrant genomic landscapes of GBM incorporating gene expression, whole genome copy number arrays and chromosomal translocations, epigenomics, whole exome sequencing and microRNA expression arrays (Verhaak, R. G., et al., Cancer Cell. (2010) 17:98-110). This study was done on a large cohort of clinically well-defined tumor specimens (>500) and non-tumor samples, and provided new insights on three key disease-perturbed pathways (McLendon, R., et al., Nature (2008) 455:1061-1068).

Although highly informative, these studies failed to appreciate the existence of GBM cancer stem cells (CSCs) that are capable of forming tumors in immune-deficient mice (O'Brien, C. A., et al., Nature (2007) 445:106-110; Singh, S. K., et al., Nature (2004) 432:396-401). As these CSCs exist in non-dividing and non-proliferative states for extended periods, physical isolation of these cells through careful selection of bona fide cell-surface proteins expressed on CSCs would be useful to characterize these populations better. Antibodies targeting the cell surface transmembrane protein CD133 have been used to isolate CSCs from bulk tumor populations, but several recent studies have suggested significant limitations of CD133 as a stand-alone CSC marker and have highlighted the need for additional cell-surface markers (Kemper, K., et al., Cancer Res. (2010) 70:719-729; Wan, F., et al., Brain Pathol. (2010) 20:877-889; Wang, J., et al., Int. J. Cancer (2008) 122:761-768; and Chen, R., et al., Cancer Cell. (2010) 17:362-375).

In view of the very short therapeutic window afforded by GBM, the need for early diagnosis is apparent, and to date, no simple significant assay that is sufficiently non-invasive to result in early diagnosis is available. In addition, the failure of conventional treatments for GBM indicates the necessity to identify individuals who will be responsive to particular types of treatment. In particular, the present invention provides a straightforward method to diagnose and a basis for assessing whether individuals who have been diagnosed with GBM will respond to TGF-β1 inhibitors.

### DISCLOSURE OF THE INVENTION

The invention provides a set of protein markers that are accessible by assaying blood samples to provide an assessment of the probability that a subject is afflicted with GBM. Thus, in one aspect, a method for assessing the probability that a human subject is afflicted with glioblastoma multiforme (GBM) which method comprises:

a) assessing the level of at least one protein selected from the group consisting of HMOX1, CD44, VCAM1, and TGFBI (BIGH3) in the blood or fraction thereof of a test subject;

b) comparing the level of said at least one of said proteins to the level of said protein in the blood or fraction thereof normal subjects;

wherein a decreased level of CD44 and/or an increased level of HMOX1 and/or a decreased level of VCAM1 and/or a decreased level of TGFBI in the test subject as compared to normal subjects indicates the probability that said test subject is afflicted with GBM.

While these proteins are easily assayed in blood, their levels in brain tissue and tumor cells may also be used as markers.

In another aspect, the invention is directed to a method for assessing the probability that a human subject is afflicted with glioblastoma multiforme (GBM) which method comprises:

assessing the level of at least one protein selected from the group consisting of ABCA1, ASPH, CA12, CADM1, CAV1, CD109, CD151, CD276, CD44, CD47, CD97, CD99, CLCC1, CRTAP, DDR2, EGFR, HMOX1, ITGA7, MGST1, MRC2, MYOF, NRP1, PDIA4, PTGFRN, RTN4, S100A10, SCAMP3, SLC16A1, SLC16A3, TGFBI, TMX1, TNC, and VCAM1 in the brain tissue, tumor cells or blood or fraction thereof of a test subject; and comparing the level of said at least one of said proteins to the level of said protein in the brain tissues, tumor cells or blood or fraction thereof normal subjects;

wherein a difference in the level in the test subject as compared to normal subjects indicates the probability that the test subject is afflicted with GBM.

In still another aspect, the invention is directed to ordered panels of reagents designed to detect these and additional proteins that have been identified as described below as indicative of the presence of GBM in a test subject.

In still another aspect, the invention is directed to a method to assess whether a subject will respond to treatment for GBM by administering an inhibitor of TGF-β1. This results from the understanding that some of the proteins that can be used to identify GBM as distinguished from normal tissue indicate an abnormality due to an enhancement of the ability of TGF-β1 to promote invasiveness.

In still other aspects, the invention is directed to a method to treat GBM by modulating the expression or activity of proteins identified as promoting invasiveness upon TGF-β stimulation and to a method to classify GBM.

### BRIEF DESCRIPTION OF THE DRAWINGS

FIG. 1 shows cross-transcriptomic characterization of cell surface proteins identified from shotgun proteomics with the tissue arrays obtained from GBM (n=228) and related diseases viz. astrocytoma (n=148) and oligodendroma (n=67). These identifications were used to develop GBM-specific membrane signature (GBMSig) comprised of 33 cell surface proteins (CSPs). Each column of the heat-map is presented as the average log 2 [tumor/non-tumor] ratios.

FIG. 2a shows principal component analysis (PCA) of REMBRANDT GBM transcriptome arrays with GBMSig proteins (n=33). The darker dots on the left represent non-tumor isolates and grey ones are GBM.

FIG. 2b shows PCA of TCGA GBM transcriptome arrays with GBMSig (n=33). The darker dots on the left represent non-tumor isolates and grey ones are GBM.

FIG. 2c shows sensitivity and specificity analysis of GBMSig (n=28) with non-identical GBM tissue arrays in TCGA (n=547 GBM and n=10 non-tumor) revealed high degree of specificities in identifying GBM populations.

FIG. 2d shows sensitivity and specificity analysis of GBMSig (n=33) with REMBRANDT tissue arrays.

FIG. 3a shows the results of d-SRM assays employed to identify unique GBMSig that can distinguish cancer stem cells (CSCs) (from Celprogen) and healthy neural stem cells (NSCs) (from Millipore).

FIG. 3b shows the validation of SRM results of GBMSig expression by an alternate method flow cytometry. An independent primary cancer stem cell obtained from GBM patient also revealed higher expressions of HMOX1, SLC16A1, but lower expression of SLC16A3 relative to healthy neural stem cells.

FIG. 4 is a heat-map showing the enrichment pattern of 21 GBMSig in tissue homogenates from individual patient tumor (n=4) compared to non-tumor isolates (n=2).

FIG. 5a shows association of GBMSig proteins with TGF-β1 signaling network.

FIG. 5b shows effects the association of GBMSig proteins with cancer invasion.

FIG. 5c shows the poor survival (p<0.003) of GBM patients (n=100) from REMBRANDT repository when GBMSig proteins were over expressed.

FIG. 6a shows ELISA assays of 21 healthy and 21 GBM plasmas for the indicated GBMSig proteins HMOX1, CD44, VCAM1 and TGFBI.

FIG. 6b shows ROC analysis (10,000×10 folds cross validation) of HMOX1, CD44, VCAM1, and TGFBI ELISA results, which offer a basis to diagnose GBM from blood analyses.

FIG. 7a shows changes in the plasma values of HMOX1, CD44, VCAM1, and TGFBI (BIGH3) at 24 hrs, 48 hrs, and 10 days after tumor resection as measured through ELISA assays.

FIG. 7b shows ROC analysis of plasma values of HMOX1, CD44, and TGFBI within 10 days after tumor resection.

FIG. 7c shows PCA analysis of plasma values of HMOX1, CD44, and TGFBI (BIGH3) at 24 hrs and 10 days after tumor resection.

### MODES OF CARRYING OUT THE INVENTION

The invention is directed to the methods and compositions that are indicated useful in diagnosis and selection of treatment method based on the nature and level of surface proteins that are characteristic of GBM as opposed to normal tissue, as well as to methods to treat GBM.

The invention herein relates to 1) cell surface GBMSig classifiers (33 cell surface proteins such as ABCA1, ASPH, CA12, CADM1, CAV1, CD109, CD151, CD276, CD44, CD47, CD97, CD99, CLCC1, CRTAP, DDR2, EGFR, HMOX1, ITGA7, MGST1, MRC2, MYOF, NRP1, PDIA4, PTGFRN, RTN4, S100A10, SCAMP3, SLC16A1, SLC16A3, TGFBI, TMX1, TNC and VCAM1) that can accurately distinguish GBM tissues from healthy tissues at both transcript and proteome level, 2) blood biomarkers among GBMSig proteins, a subset of which was validated by d-SRM and ELISA, 3) disrupted TGF-β network components represented by key GBMSig proteins in GBM and 4) representative cell surface markers for GBM cancer stem cells (GCSCs).

Aberrant expression and activity of cell-surface proteins are hallmarks of most cancers. These proteins occupy a strategic location between the cell and its microenvironment and can perceive signals that emanate from both exofacial and cytoplasmic ends of the membrane. Thus, aberrant expressions of these proteins on the cell surface disrupt the normal activities of a cell and influence neoplastic transformation. The differences in the expressions of cell-surface proteins between healthy and cancerous tissues can both serve as cancer markers and provide information for developing targeted therapies. Those cell-surface proteins that are cleaved and shed into the blood are useful as diagnostic blood markers.

The present inventors analyzed cell surface proteins in GBM through comparative analysis of a representative GBM CSCs, healthy NSCs, and bulk tumor cell populations exemplified by U87 and T98 cell lines. Cell-surface proteomics data were combined with the large-scale GBM tissue transcriptomic array analyses from REMBRANDT and TCGA tumor compendiums. This integrative approach resulted in a GBMSig comprising 33 cell surface proteins that characterize of GBM tissues.

The cell-surface proteins from four cell lines that have relevance in GBM were analyzed. These include two cell lines that represent bulk tumor populations, U-87 and T-98, a representative healthy NSC line (positive for putative stem cell markers tub iii, oct-4, sox-2 and CD133) and a GBM CSC line (positive for CD133 expression). To enrich for typically low abundance cell-surface proteins, the membrane impermeable sulfo-NHS-SS-biotin strategy was used to capture cell-surface proteins from intact cells. Cell-surface composition of each cell line appears significantly different from the others, which suggests that these four cell lines may be functionally different as well or the heterogeneity might just reflect the increased mutational process fundamental to all cancers.

Captured cell surface proteins were subjected to high resolution mass spectrometry in triplicates and the proteins were identified using the Global Proteome Machine [(the GPM) (located on the World Wide Web at: theGPM.org)] with minimum log expectation scores of <10⁻³. A total of 868, 813, 541 and 564 non-redundant proteins were identified from U87, T98, NSC, and CSC populations, respectively. The transmembrane prediction algorithm TMHMM was employed to identify these transmembrane (TM) proteins from the total cell-surface protein preparation, leading to the identification of 157, 154, 98 and 80 TM proteins in U-87, T-98, NSCs and CSCs, respectively. Overall 273 different TM proteins were identified from all four cell lines. Among TM proteins identified, there were 53 CD markers, 98 multi-TM domain containing cell-surface proteins, the latter of which are underrepresented in whole-cell proteomic datasets because of their hydrophobicity and limited cellular abundances.

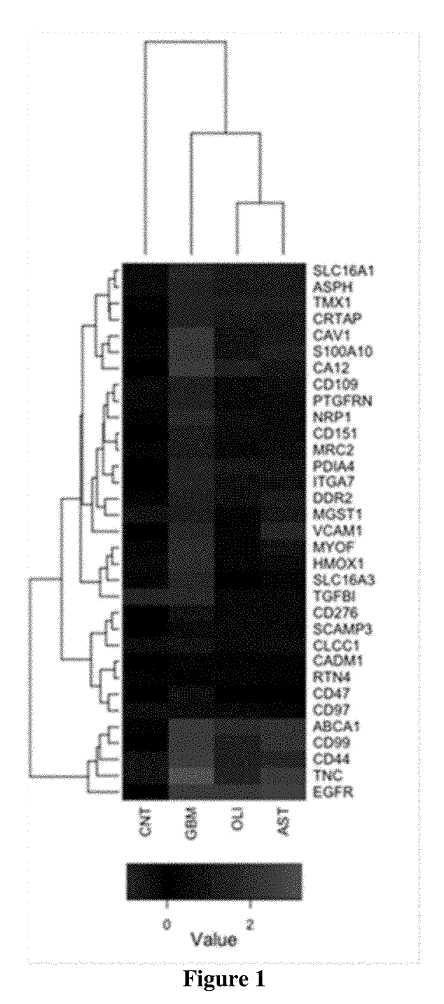

The presence of differentially expressed transcripts between tumor and non-tumor regions of the brain was evaluated by integrating cell-surface proteomics data with the transcriptome compendium from the REMBRANDT tissue source (Madhavan, S., et al., Mol. Cancer Res. (2009) 7:157-167). Out of 270 cell-surface TM proteins identified from cell surface proteomics study, information for 202 (532 independent probes) was found as corresponding transcripts in REMBRANDT. Expression values for these transcripts were log 2 transformed, and a minimum of 2-fold average expression (FDR<0.05) relative to non-tumor brain tissues were used as cut-off for expression analysis. Among 202 transcripts of cell-surface proteins, 155 of them were unregulated and 47 were down-regulated between 228 tumor and 9 non-tumor regions of the brain. To identify GBM-related cell-surface protein expression changes, transcripts also differentially expressed in other brain diseases such as astrocytoma (N=148 tumors) and oligodendroma (N=67 tumors) as mentioned in REMBRANDT were filtered out. A GBM-membrane unique signature (GBMSig) comprised of 33 cell-surface proteins was obtained as shown in FIG. 1.

To test the performance of GBMSig in diverse GBM subjects, TCGA gene expression arrays that were built on GBM specimens (N=547 GBM tumor samples and 10 healthy brain tissues) distinct from the ones in REMBRANDT were evaluated. Twenty-eight (28) of 33 GBMSig transcripts in TCGA were also differentially expressed between GBM and non-tumor regions of the brain.

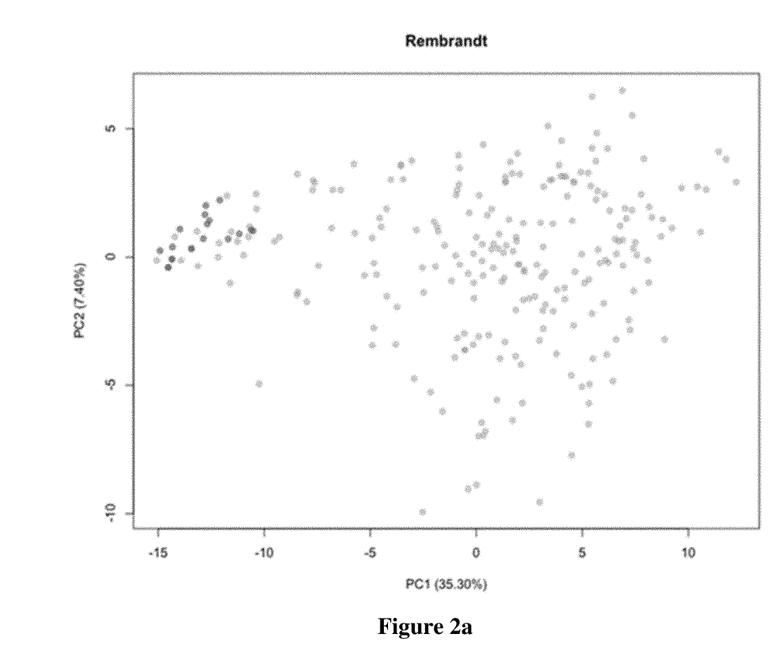

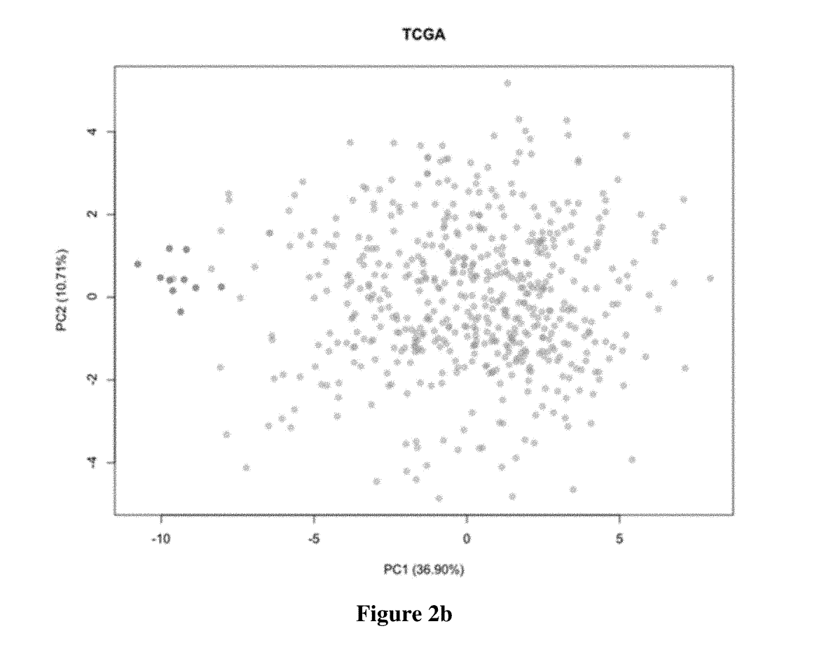

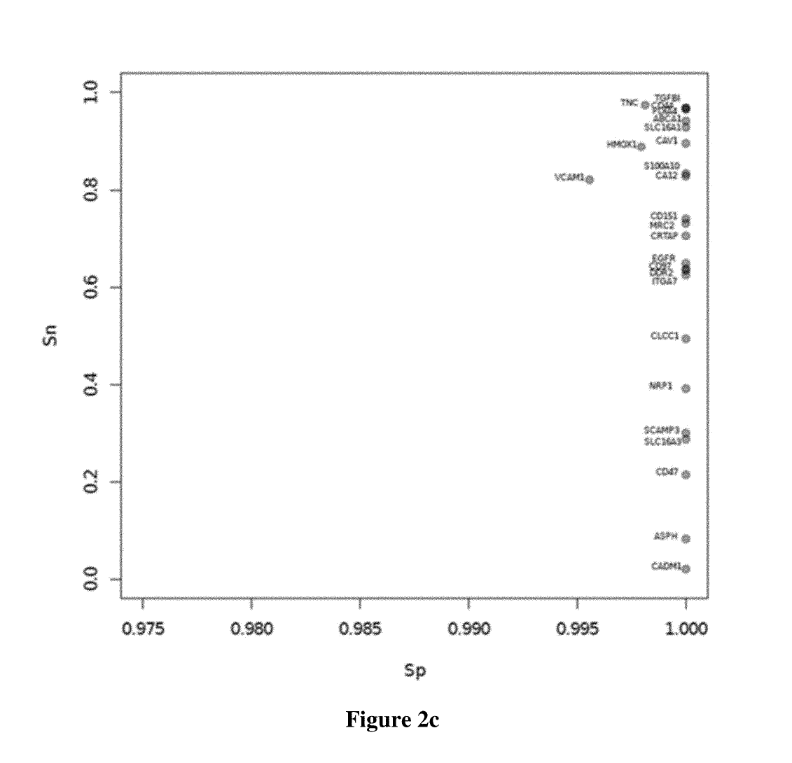

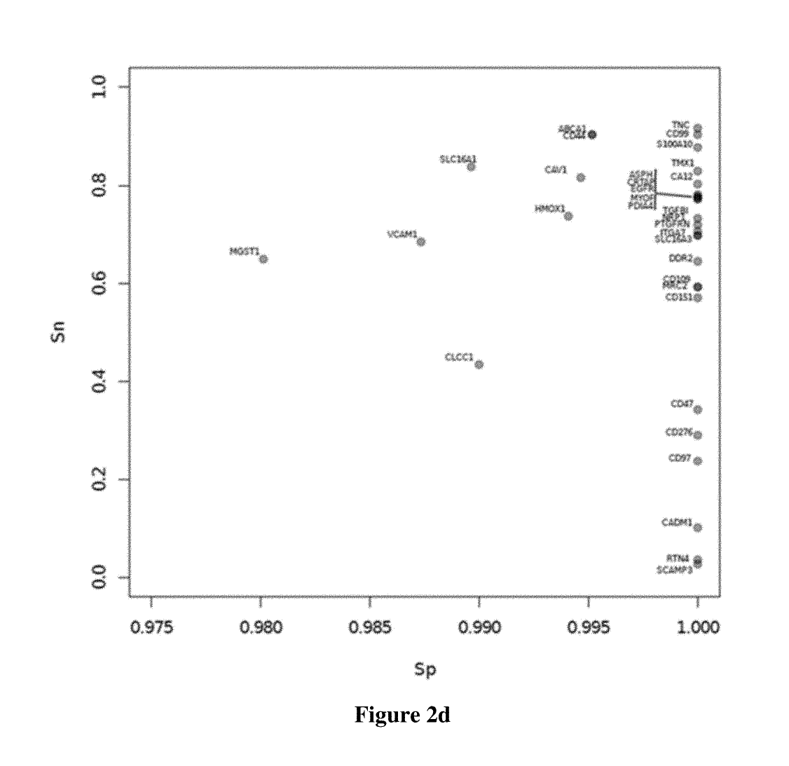

To assess the discriminating power of GBMSig, the classifier was (i.e., the 33 proteins of GBMSig) evaluated by support vector machines (SVM)-supervised learning models. After 10-fold cross validation(CV) and fitting the model on training dataset (REMBRANDT) the hyperparameters (parameters tuned after 10-fold CV) were identified and the discerning capabilities of the classifier on validation set (TCGA dataset) was predicted. This resulted in 99.85% sensitivity, 75% specificity, 99.54% positive predictive value, and 90.69% negative predictive value for the classifier. Principal component analysis (PCA) of the classifier and individual specificities and sensitivities on both test set and validation set is presented in FIGS. 2a-2d. GBMSig effectively distinguishes GBM from non-tumor counterparts.

To explore the utility of GBMSig for stratifying tumors into discrete subtypes, additional sets of tumor tissues (N=216) in TCGA pre-classified as classical (N=64), mesenchymal (N=59), proneuronal (N=59) and neuronal (N=34) were tested. Relative rank-orders of each gene across the pre-stratified GBM tissues (N=216) were determined from their respective Z-scores. Each GBMSig gene with its highest Z-score in a given GBM subtype was assessed by ROC analysis for its discriminatory ability to identify a dominant GBM subclass. There were 9 GBMSig proteins viz. ASPH, SCAMP3, CLCC1, and CADM1 representing proneuronal; CD44, ITGA7, and EGFR representing classical; CAV1 and TGFBI representing mesenchymal subtypes with high degree of specificity (>80%).

### Review of Examples

Initially the cell-surface composition of various GBM cell lines including U87, T98, CD133⁺CSC (Celprogen) and a NSC line (Millipore) were examined by high resolution mass spectrometry that led to the identification of cell-surface proteins especially those with transmembrane domains. The sequence of peptides showed the mass spec compatibility of the peptides required to set-up SRM assays. Integrated cell-surface proteomics data was integrated with large scale GBM tissue transcriptome repositories in REMBRANDT (228 GBM and 9 non-tumors) and TCGA (547 GBM and 10 non-tumors) repositories. From these integrated analyses, a GBM membrane signature (GBMSig) was developed. It is composed of 33 cell-surface transmembrane proteins that can accurately distinguish GBM tumors from normal tissue with a high degree of sensitivity (97.30%, 10 fold CV), specificity (95%, 10 fold CV), and precision (99.56%, 10 fold CV) on training dataset (REMBRANDT, tumor=228, non-tumor=9). After fitting the SVM model with training dataset (REMBRANDT) and locking down the hyperparameters a high degree of sensitivity (99.85%, 10 fold CV) and specificity (75%, 10 fold CV) was obtained from validation dataset (TCGA, tumor=547; non-tumor=10) with 99.54% positive predictive value and 90.69% negative predictive value from 10 independent iterations.

PCA analysis based on differential GBMSig expression between tumors and non-tumors in REMBRANDT and TCGA datasets also revealed comparable degrees of separation as shown in FIGS. 2a-2d highlighting the robust predictive power of GBMSig panel in diagnosing GBM along multiple datasets. d-SRM targeted proteomics assays were developed, permitting multiplexing capability and higher throughput in sample analysis, and also permitted detection of otherwise low abundant CSPs in biological isolates. Overall, the enrichment pattern of 21 of the 33 GBMSig uniquely represented GBM tissue as demonstrated further by Spearman clustering and in the representative PCA analysis. Alternate validation of 4 GBMSig proteins shed into plasma viz. VCAM1, HMOX1, CD44, and TGFBI with 21 GBM and 21 healthy plasmas (age and gender matched) by ELISA also revealed high degree of sensitivity and specificity for GBM vs. healthy subjects

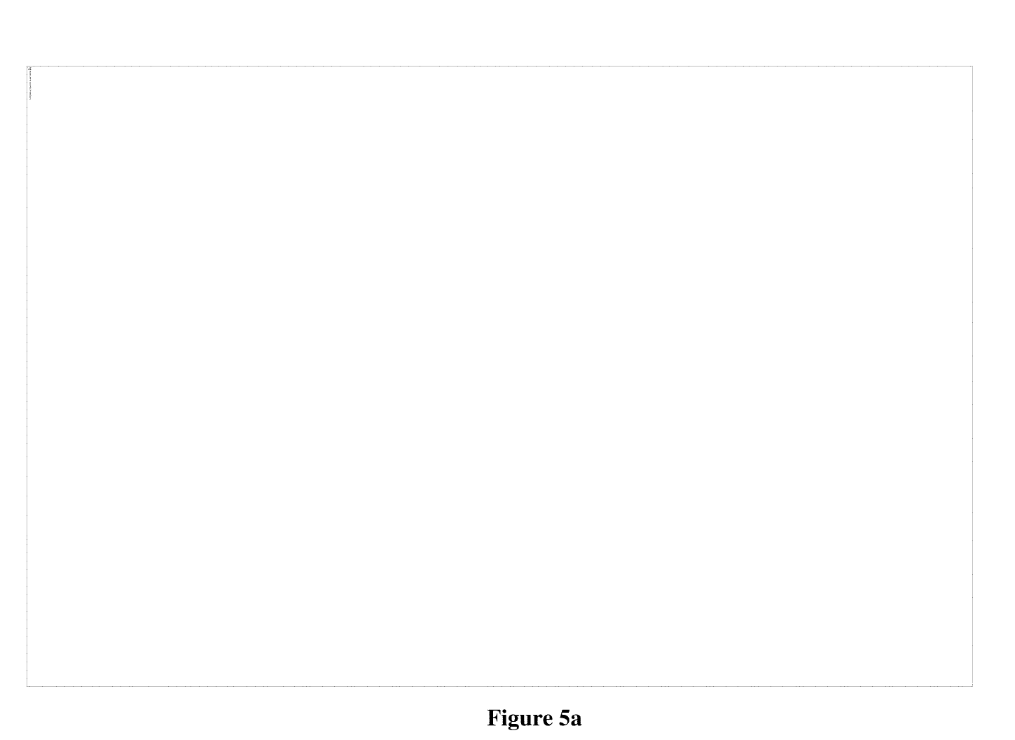

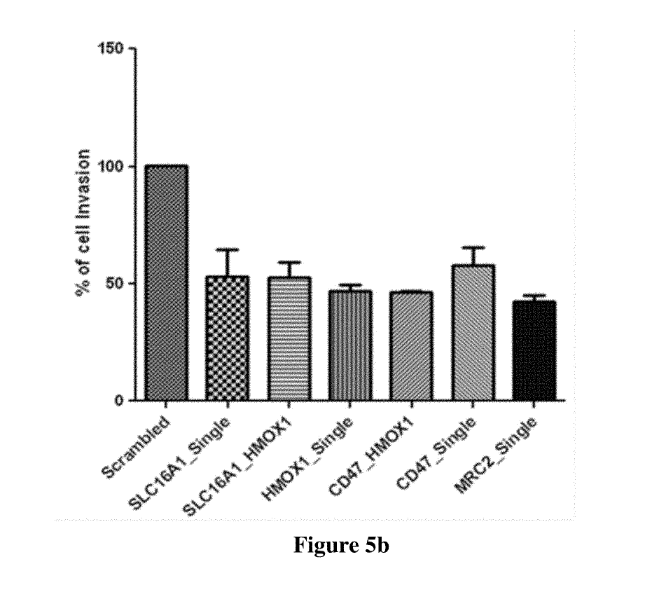

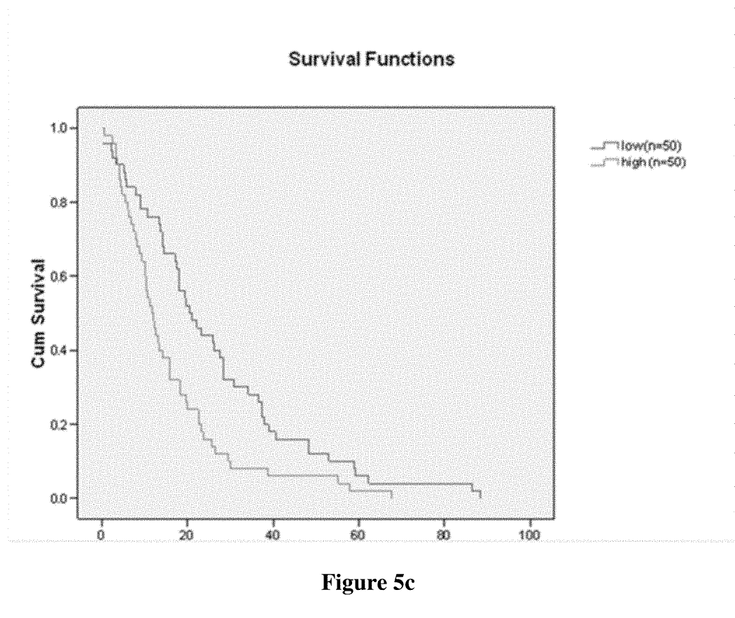

Tissue SRM analysis showed that a number of GBMSig proteins—possibly deregulated as a consequence of the disease were co-overexpressed with TGFBI a TGF-β inducible protein, indicating a putative regulation of these GBMSig proteins through TGF-β signaling. Novel TGF-β responsive elements were identified among GBMSig through experimental validation. Modular roles of 19 GBMSig proteins were demonstrated in TGF-β responsiveness through in vitro analysis using the U87 cell line. U87 cells were treated with TGF-β1 or its inhibitor alone or sequentially with inhibitor followed by TGF-β1. Changes in expressions of GBMSig proteins following such treatments were measured by SRM assays. The results indicate the association of a subset of GBMSig proteins with TGF-β1 signaling that has not been disclosed previously. These results are shown in FIG. 5a.

A subset of these novel TGF-β responsive proteins viz. SLC16A1, HMOX1, MRC2, CD47, SLC16A3 and CD97 were further investigated to characterize TGF-β responsiveness among GBM cells relative to healthy NSCs as shown in FIG. 5b. U87 cells treated with siRNA for the indicated proteins were allowed to migrate towards TGF-β1 gradient through basement membrane (Cell Biolabs Inc.). Invaded cells were analyzed through colorimetric assay. Results from three independent experiments were averaged and normalized to non-targeting siRNA pools (scrambled). Loss of cell migration following siRNA mediated inhibition of SLC16A1, MRC2, and HMOX1 is similar to that of known invasive marker CD47.

Isogenic cell lines of U87 where key proliferative genes such as EGFR and EGFRVIII are overexpressed alone or in combination with PTEN were tested for molecular responsiveness of isogenic cell lines towards TGF-β treatment. In EGFR and EGFRVIII isogenic cell lines, there was elevated surface expression of SLC16A1 and HMOX1 in response to TGF-β treatment. PTEN expression, however, inhibited this effect, indicating possible involvement of a tumor suppressor PTEN in modulating the surface expression of these proteins in GBM. On the other hand, MRC2 and CD47 were up regulated in response to TGF-β treatment when PTEN was overexpressed.

SN143 tumor-derived GCSC populations exhibited TGF-β responsiveness different from the isogenic cell lines from U87. They showed a 30%-increase in surface expression of HMOX1 in response to TGF-β-inhibitor treatment relative to TGF-β treatment, though an increase in expression of MRC2 in response to TGF-β treatment over its inhibitor was similar to that of U87 cell lines. As HMOX1 has been known to protect cells during oxidative damage and thus by regulating the expression of this protein, a GCSC may escape damage caused by therapeutic agents. The observed increase in expression of HMOX1—the cell-surface protein which was found to be enriched on proliferating SN143 cells—may be related to this defense response.

These results show the elasticity of cancer cells is maintained through the recruitment of multiple cell surface proteins that have complementary activities. The responsiveness of NSCs to TGF-β was strikingly different from that of GBM cell lines and from that of SN143-tumor derived GCSCs. In NSCs, there were no significant changes in the cell-surface expression of SLC16A1, HMOX1, MRC2, CD47, SLC16A3 and CD97 in response to TGF-β treatment. This result may favor an inactivated and unperturbed TGF-β network in healthy NSCs that is poised to dampen neurogenesis and proliferative capabilities as mentioned in earlier reports. Additionally, TGF-β-inhibitor treatment of NSCs resulted in increased expression of MRC2, CD47, SLC16A3 and CD97, i.e., GBMSig proteins that were inhibited in GBM cells following TGF-β-inhibitor treatment. While TGF-β-inhibitor responsiveness of primary SN143 cells was likely mediated through HMOX1 overexpression, it was MRC2 that exhibited similar effects in NSCs. The results indicate that SLC16A1, MRC2, and HMOX1 are important mediators of TGF-β signaling in cancer cells, the regulation of which is distinct from the molecular responsiveness of healthy NSCs—possibly due to differences in operational framework of TGF-β networks in healthy and cancer cells. The results also show siRNA-mediated inhibition of SLC16A1, HMOX1, and MRC2, resulted in reduced cell invasion (>50% in comparison to scrambled siRNA treated cells) similar to that of a known invasive marker CD47, pointing to direct involvement of these proteins in GBM invasion and TGF-β responsiveness. It is, therefore, likely that the invasive nature of SLC16A1, HMOX1, CD47 and MRC2 and the overexpression of these proteins on GCSCs may enable these cells to contribute to metastasis in response to TGF-β1 and therefore negatively impact patient survival. Thus, characterizing expression of these proteins or inhibiting their activity would be an effective treatment. Survival analysis of TCGA datasets support this notion as patients co-expressing five GBMSig proteins viz. SLC16A1, HMOX1, MRC2, CD47 and SLC16A3 revealed poor survival by 30% (p<0.08) while 10 GBMSig proteins viz. CA12, MRC2, CD44, TNC, SLC16A1, S100A10, HMOX1, ITGA7, SLC16A3, and CLCC1 revealed poor survival by 50% (p<0.003) in REMBRANDT dataset. FIG. 5c shows the poor survival (p<0.003) of GBM patients (n=100) from REMBRANDT repository when GBMSig proteins were over expressed.

The results from these experiments lead to the following advances. First, there is for the first time available a method for early diagnosis of GBM that is non-invasive and based on a blood test. The results below show that four of the members of the GBMSig proteins: CD44, HMOX1, VCAM1, and TGFBI are present in altered levels in the plasma of GBM subjects as opposed to healthy subjects. It is also likely, and part of the invention, that remaining members of the GBMSig proteins will have altered concentrations in the blood of GBM subjects as compared to healthy subjects. Panels with orderly arrays of reagents for the detection of each of these blood markers, which can be packaged and used as a kit, also will find use in diagnosis.

The additional markers that may occur in blood include DDR2, PDIA4, CADM1, ITGA7, MRC2, MYOF, NRP1, RTN4, TNC, SCAMP3 and CD47.

Second, a number of the GBMSig proteins were identified as upregulated by TGF-β stimulation. These proteins appear to enhance the effect of TGF-β in promoting invasiveness. Thus, GBM tumor tissue that is obtained from subjects that have high levels of these proteins indicate that the subject is a promising candidate for therapy based on administration of TGF-β inhibitors. These proteins include SLC16A1, HMOX1, MRC2 and CD47.

Third, as the proteins indicative of enhanced response to TGF-β promote invasiveness, therapies that result in decrease in expression of these proteins or an inhibition of their activity are useful in treating GBM. Such methods include the use of expression inhibitors such as siRNA, antisense constructs, and the like, and methods to inhibit activities include administering binding agents for the proteins themselves, such as antibodies, aptamers, antibody mimics and the like.

Fourth, certain of the GBMSig proteins are shown to be characteristic of various forms of GBM. Thus, as shown in Example 6, mesenchymal, classical/proliferative, and pre-neuronal subtypes of GBM can be distinguished based on the expression patterns of specific subsets of these proteins.

Preparation A

Development of SRM Assays for GBMSig

To evaluate the role of GBMSig as protein biomarkers in GBM tissues and blood, SRM assays (Aebersold, R., et al., Mol. Cell Proteomics (2013) 12:2381-2382) were developed. A total of 70 cell-surface protein (CSP) peptide representatives from the 33 GBMSig proteins were used for d-SRM assay development—approximately 2 for each protein. Representatives of synthetic peptides labeled (¹³C¹⁵N) C-terminally with either lysine (K) or arginine (R) that act as surrogates of endogenous peptides were subjected to collision energy (CE) optimization to maximize the release of trapped energy from each peptide bond. Three parental (Q1) charges (+2, +3, and +4) and two daughter (Q3) ion charges (+1 and +2) of peptides were tested in all feasible combinations for assay optimization; the Q1/Q3 transition-CE combination that demonstrated highest abundance and were minimally affected by interfering ions was finally selected for assay validation. In the final SRM method the best performing peptide with a minimum of three transitions were used for quantitation. This targeted approach improved the sensitivity and specificity of detecting CSPs, which are typically at low abundance, in these biological isolates.

The following examples are intended to illustrate but not to limit the invention.

### Example 1

### Blood Secreted GBMSig Proteins for GBM Diagnosis

Four GBM plasmas were analyzed for circulating GBMSig by SRM mass spectrometry. Fourteen of 33 GBMSig proteins were detected independently in triplicate SRM runs. Four circulating GBMSig proteins HMOX1, CD44, VCAM, and TGFBI (BIGH3) were also evaluated by ELISA. Using 42 plasma samples (21 healthy and 21 GBM, age and gender matched) statistically significant differences were observed in the concentrations of these proteins. Comparing healthy plasma vs. GBM plasma, the concentrations were:

for CD44: 149.31 healthy versus 75.09 ng/ml GBM, (p<3.69E-08, two-tailed),

for HMOX1: 10.70 healthy versus 17.52 ng/ml GBM, (p<9.21E-05, two-tailed),

for VCAM1: 583.22 healthy versus 436.40 ng/ml GBM, (p<0.02, two-tailed), and

for TGFBI: 2482.51 healthy versus 931.74 ng/ml GBM, (p<5.68E-10).

Thus, concentrations of CD44, VCAM1, and TGFBI were decreased in GBM plasma and HMOX1 is increased.

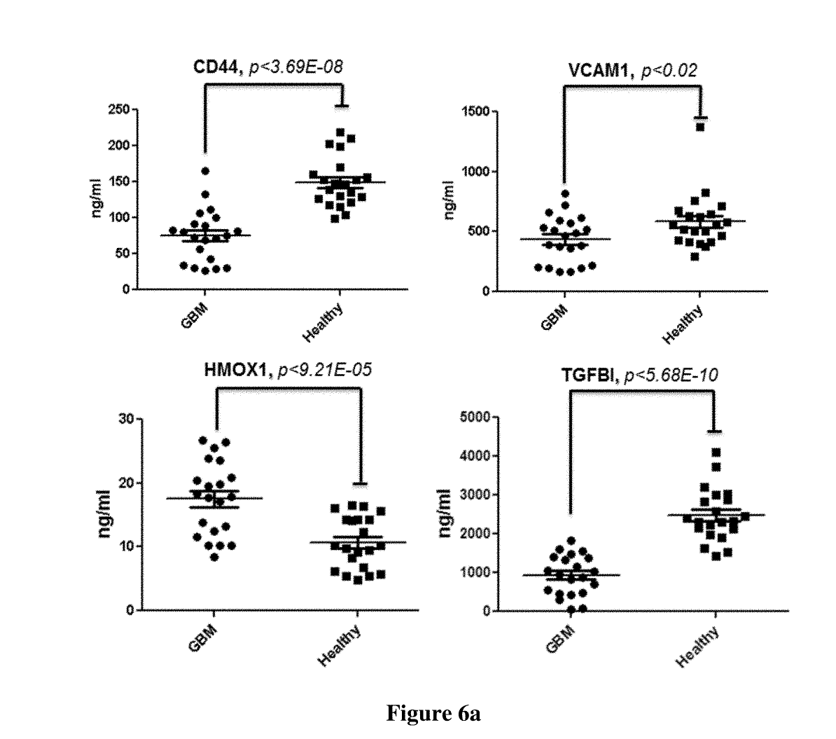

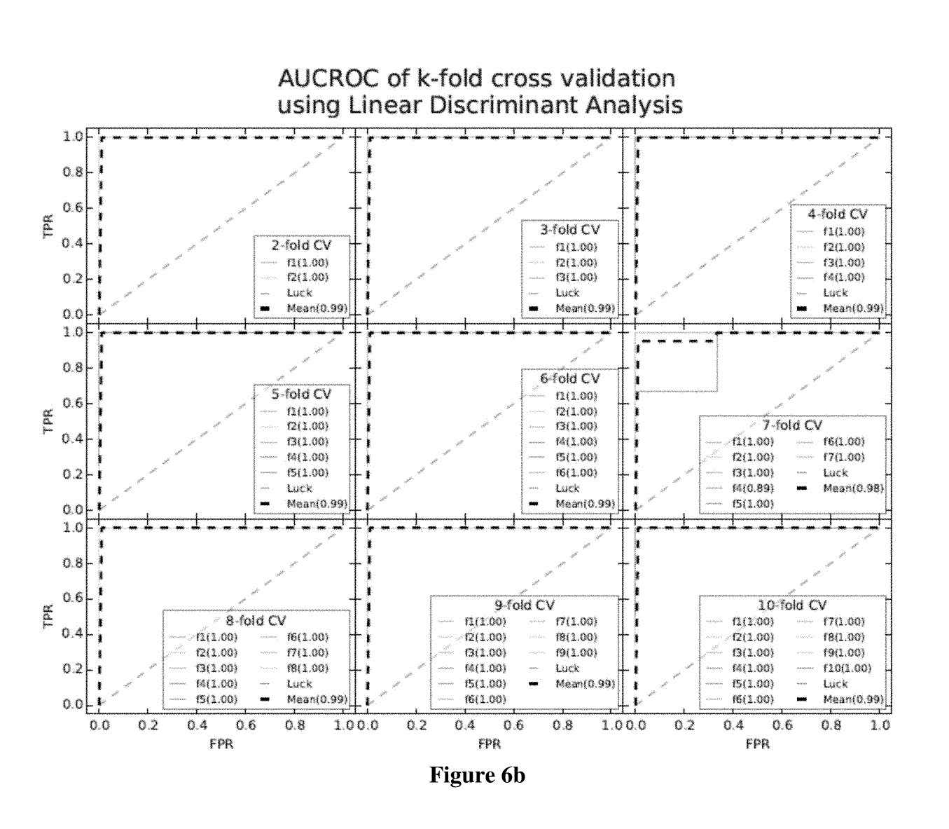

These results are shown in FIG. 6a. FIG. 6b shows ROC analysis (10,000×10 folds cross validation) of HMOX1, CD44, VCAM1, and TGFBI ELISA results, which offer a basis to diagnose GBM from blood analyses.

Each sample was analyzed in duplicate. ROC analysis revealed areas under the curves (AUCs) of 0.934 for CD44, 0.831 for HMOX1, 0.685 for VCAM1, and 0.982 for TGFBI. A combined mean AUC of 0.99 in 10,000×10 fold CV for CD44, and HMOX1 was found. The ELISA results also indicated good agreement between the effect size (>10.81) and the sampling method (power>0.8) for the current procedure.

### Example 2

### Common TGF-β Response Among GBMSig Proteins

A number of GBMSig proteins exhibited moderate to high correlation with higher levels of co-expression with TGFBI when either GBM or normal tissues are assayed. TGFBI is a TGF-β inducible protein that plays important role in cancer invasion. TGF-β1 is an inducer of epithelial to mesenchymal transition (EMT) and plays cardinal role in several aspects of GBM biology including the local metastasis of tumor cells, maintenance of cancer stem cell niche and therapeutic resistance of cancer cells. GBMSig protein levels that correlate with stimulation by TGF-β1 in a subject indicate that inhibitors of TGF-β1 may be beneficial in treating GBM in such subjects.

To identify markers for subjects that may benefit from this treatment, astrocytoma cell line U87 was serum starved overnight and treated with 10 ng/ml TGF-β1 for 40 hrs. TGF-β treatment increased the C-terminal phosphorylation of SMAD2 in comparison to cells grown in serum-free media, suggesting the activation of TGF-β1 signaling. However, as serum contains many essential elements and growth factors, the effect of serum starvation on cells might not be specific to the inhibition of TGF-β signaling. Therefore, we employed a TGF-β-inhibitor (SB 431542) known to interfere with the C-terminal phosphorylation of SMAD2. Cells grown in normal media (DMEM+10% FCS) supplemented with TGF-β1-inhibitor dampened or diminished C-terminal phosphorylation of SMAD2 similarly to what was observed for cells grown in serum-free media. Thus in subsequent SRM analysis, TGF-β-inhibitor was used instead of serum starving.

Out of 31 d-SRM assays conducted using the U87 cell line, 11 GBMSig proteins including TGFBI exhibited at least two fold higher expression following TGF-β treatment relative to cells treated with TGF-β-inhibitor alone. These proteins are

TGFBI (10.54 fold±3.01 SEM),

ITGA7 (9.45 fold±5.33 SEM),

TNC (6.55 fold±1.19 SEM),

DDR2 (3.53 fold±0.83 SEM),

MRC2 (3.06 fold±0.164 SEM),

MGST1 (2.77 fold±0.32 SEM),

CLCC1 (2.26 fold±0.468 SEM),

PTGFRN (2.18 fold±0.184 SEM),

CRTAP (2.12 fold±0.452SEM),

CD109 (2.09 fold±0.61 SEM), and

SLC16A1 (2.05 fold±0.20 SEM).

These 11 proteins were also overexpressed when TGF-β-inhibitor treated cells were retreated with TGF-β. The association of these 11 proteins with SMAD2 dependent TGF-β signaling has not been disclosed previously.

There were 8 additional GBMSig proteins viz. CD47, VCAM1, MYOF, ABCA1, CD44, S100A10, CA12, and SLC16A3 that exhibited positive enrichment (>1.3 fold over inhibitor treatment) on TGF-β treatment vs. TGF-β-inhibitor treatment, but 4 GBMSig proteins viz. ASPH, NRP1, CD276, and HMOX1 were relatively reduced in expression following TGF-β treatment and 8 GBMSig proteins viz. CD97, SCAMP3, PDIA4, CD99, ABCA1, TMX1, RTN4, and CD151 remained largely unchanged following TGF-β treatment in comparison to inhibitor treatment.

In an alternate assay, six GBMSig proteins viz. SLC16A1, MRC2, CD47, SLC16A3, HMOX1, and CD97 were tested as downstream factors of TGF-β signaling by flow cytometry. TGF-β or TGF-β inhibitor treated intact U-87 cells were analyzed by flow cytometry and the ratio of the respective GBMSig expression in response to TGF-β1 over its inhibitor was obtained. The ratio of protein on the cell-surface TGF-β over TGF-β inhibitor was found to increase by the following amounts in each case.

CD47: 40% (p<3.68E-08),

SLC16A3: 30% (p<4.72E-09),

MRC2: 25% (p<6.43E-08),

SLC16A1: 20% (p<4.44E-06),

HMOX1 20% (p<5.45E-06) CD97 essentially no change (1.1 fold increase).

The discrepancy in HMOX1 expression may be individual protein-specific and related to differential partitioning of proteins on the cell surface in comparison to total internal pools. Thus, a subset of GBMSig molecules that enhance TGF-β signaling has been identified: CD47, SLC16A3, MRC2, SLC16A1, and HMOX1.

### Example 3

### Role of Certain GBMSig Proteins in TGF-β1-Mediated Invasive Response

As TGF-β1 is an inducer of the EMT process, the subset of GBMSig that were identified in Example 2 as TGF-β1 responders may contribute to the invasiveness of astrocytoma cells. TGF-β responsive GBMSig genes were silenced using si-RNA in U87 cells and the ability of cells in which these genes were silenced to invade through extracellular matrix was assessed. siRNAs were directed against SLC16A1, HMOX1, MRC2, and CD47 individually or in combinations (SLC16A1+HMOX1 and CD47+HMOX1). The efficiency of siRNA mediated gene silencing was evaluated by both qPCR-at the transcript level and by flow cytometry on the cell surface. Greater than two fold reduced expression of the target genes in comparison to non-targeting RNAs was found. To account for any effect on the cell viability before and after siRNA treatments, viability was tested with calcein AM assay. No change in cell viability was found.

To evaluate the impact of gene silencing on migration and invasion, siRNA or non-targeting RNA treated cells were seeded in transwell chambers and the degree of cell invasion was evaluated as percentage of cells invaded by silenced vs. non-silenced cells. Silencing of SLC16A1, HMOX1 and MRC2 resulted in 52.88%±9.70 SEM, 46.76%±2.27 SEM, and 42.26%±2.19 SEM reduction of cell invasion respectively, similar to cells where the known invasive protein CD47 was silenced (57.74%±6.32 SEM reduced cell invasion).

Combinatorial silencing of SLC16A1+HMOX1 and HMOX1+CD47 also revealed an impact on cell invasion (52.28%±5.35 SEM and 46.55%±0.18 SEM respectively).

In summary, these results show SLC16A1, HMOX1, and MRC2 play crucial roles in the migration and invasion of GBM cells. These are also expressed on GBM cancer stem cells (GCSCs from commercial source as well as GCSCs from SN143 tissue).

### Example 4

### TGF-β1 Response in GBM Cell Lines Vs. Healthy Neural Stem Cells

Commonly mutated GBM genes include EGFR, EGFRVIII, and PTEN. Four isogenic cell lines of U87 in which these genes are expressed alone or in combinations of EGFRVIII and PTEN or EGFR and PTEN via a stably integrated retroviral vector were used (Gini, B., et al., Clin Cancer Res. (2013) 19:5722-5732).

There was an increase in the cell-surface expression of CD47, SLC16A3, MRC2, HMOX1 and SLC16A1 in response to TGF-β in all U87 isogenic cell lines similar to that of the parental cell line. However, in U87 isogenics overexpressing EGFRVIII or EGFR, an increase in expression of SLC16A1 of 1.75 fold±0.029 SEM and 1.5±0.028 SEM respectively was observed and of HMOX1 the increase was 2.13 fold±0.03 and 2.69 fold±0.06 SEM respectively compared to the parental cell line. Expression of both proteins was found to be reduced (1.19 fold±0.020 SEM for SLC16A1 and 1.48 fold±0.03 SEM for HMOX1) in EGFRVIII+PTEN cells and also in EGFR+PTEN cells (0.724 fold±0.003 SEM for SLC16A1 and 1.058 fold±0.01 SEM for HMOX1) possibly highlighting the fact that cell surface expression of these proteins is regulated through the expression of a phosphatase PTEN.

Higher expression of SLC16A3 (1.3 fold±0.025 SEM) and CD97 (1.51 fold±0.007 SEM) in EGFRVIII+PTEN compared to EGFRVIII (0.97 fold±0.027 SEM for SLC16A3 and 0.75±0.010 SEM for CD97) alone was found.

The expression of the selected GBMSig proteins responsive to TGF-β viz. SLC16A1, HMOX1, MRC2, SLC16A3, CD47, and CD97 was also tested in primary GBM cells from SN143 tumor tissues in the presence of TGF-β or its inhibitor by flow cytometry.

In primary GBM cells (obtained from SN143 tissue) lower expression of SLC16A1 (0.79 fold±0.009 SEM) and HMOX1 (0.77 fold±0.012 SEM), higher expression of MRC2 (1.62 fold±0.01 SEM) when treated with TGF-β1 was found compared to its inhibitor. Healthy NSCs showed lower expression of MRC2 (0.19 fold±0.034 SEM) and CD47 (0.61 fold±0.003 SEM) in response to TGF-β-inhibitor treatment compared to TGF-β1 treatment. SLC16A1, HMOX1 and CD97 exhibited little or no effect to TGF-β1 or inhibitor treatment highlighting distinct responsiveness of TGF-β1 signaling in healthy cells. This indicates that there is distinctiveness in TGF-β responsiveness among different GBM cells and healthy NSCs, and that key genes viz. EGFR, EGFRVIII, and PTEN altered in GBM can create further heterogeneities in TGF-β responsiveness as observed through the expression of various GBMSig proteins.

### Example 5

### Cell-Surface Protein Expression in NSCs and GCSCs

A growing body of evidence indicates that NSCs or their progenitors can undergo mutational changes and give rise to GCSCs with sustained self-renewal capabilities to propel tumor growth, drug resistance and recurrence. Differentially expressed GBMSig proteins between NSCs and GCSCs serve as cell-surface markers to distinguish these populations.

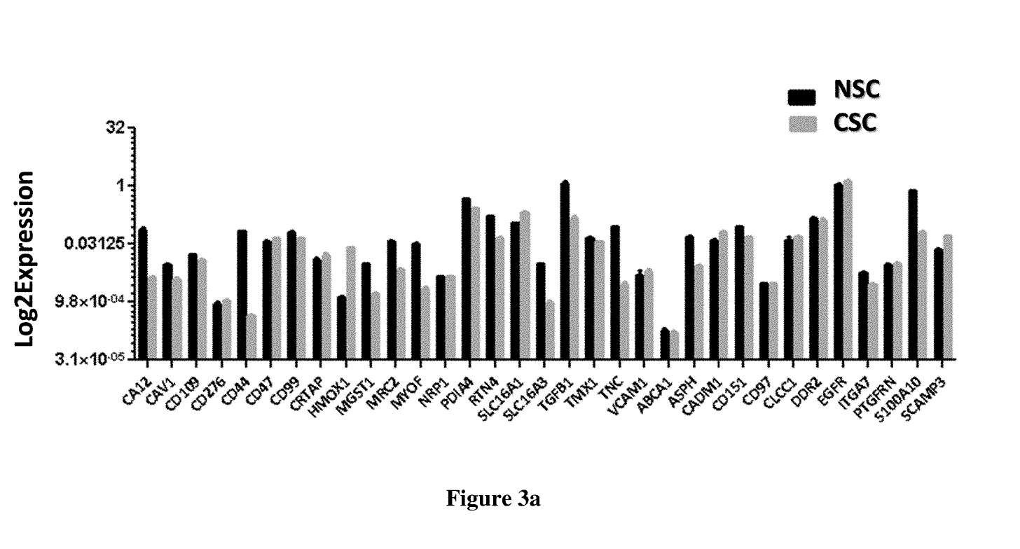

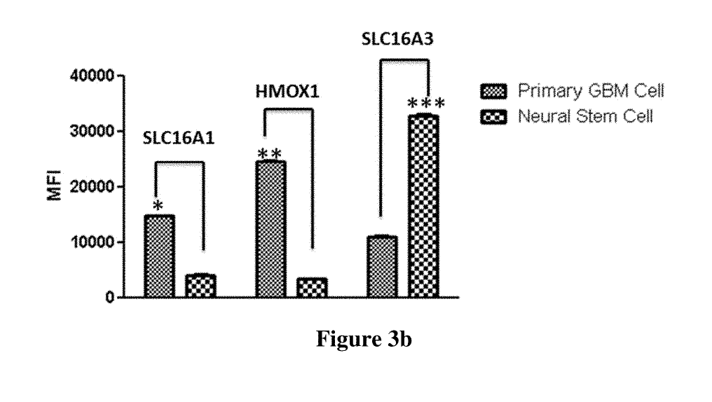

Equal quantities (5.7 μg) of cell lysates from NSCs and GCSCs were enzymatically digested and clarified, and spiked with equal quantities of SRM peptide standards (labeled C-terminally with ¹³C¹⁵N K/R) for SRM analysis. To increase the sensitivity and specificity of detection of GBMSig in cell lysates, we developed dynamic-SRM assays (d-SRM) by determining the chromatographic retention time (RT) for each peptide in prior runs using respective cell lysates. Presence of surrogate labeled peptides (C-terminal ¹³C¹⁵N K/R) in the lysates, which were co-eluted with endogenous peptides, ensured the quality and precision of assays. The peak areas of surrogate peptides and endogenous peptides were quantified through skyline and presented as a ratio of H (surrogate)/L (endogenous). Each cell type was analyzed four times and the results from these runs were averaged and shown in FIG. 3.

Out of 33 GBMSig proteins quantified through d-SRM assays, 22 of them exhibited differential patterns of expression between GCSC and NSC cells.

Four GBMSig proteins viz. SLC16A1, HMOX1, MRC2, and SLC16A3 exhibiting differential expression between GCSC and NSC cells, were further validated by flow cytometry. Intact NSC and GCSC cells were labeled with appropriate primary antibodies, and the bound antibodies were detected by FITC or PE conjugated secondary antibodies. Mean fluorescence intensities (MFI) were calculated from four replicates of each antibody type after isotype subtraction, and presented as mean values±S.E. of mean difference (SEM).

As noted above, SLC16A1, HMOX1, MRC2, and SLC16A3, were all found to be highly expressed on CSCs compared to NSCs. GCSC cells expressed SLC16A1 and HMOX1 at 26- and 8-fold higher levels, respectively, in comparison to NSC cells (p<0.001). These data are in good agreement with d-SRM assays, which also indicated higher expression of SLC16A1 and HMOX1 in GCSCs over NSCs. Reduced expressions of SLC16A3 and MRC2 on the surface of NSCs in comparison to GCSCs were evident from flow cytometry analysis.

The distinctiveness in quantitative measurement of the cell-surface proteins from two alternate sources such as cell lysates and cell surface may be related to additional regulation in subcellular partitioning of these molecules on the surface of GCSC cells.

Also examined was the potential of GBMSig proteins as potential GCSC markers on primary GBM cells, distinct from cancer stem cells from commercial source. A subset of GBMSig proteins including SLC16A1, HMOX1, MRC2, CD47, SLC16A3, and CD97 were further evaluated for surface expression levels in relation to stem-like properties of primary GBM cells. These cells were isolated from SN143 tissue (also used for targeted tissue and serum proteomics) and maintained in stem-cell mimicking conditions to enrich GCSC populations. For GCSCs grown in stem-cell enrichment media, an increase in the expression of the stem-cell marker, nestin, was observed in over 80% of the GCSC populations. Nestin enrichment on GCSCs was similar to that of NSCs.

In a parallel experiment, proliferating SN143 cells grown in stem cell-enrichment media were allowed to differentiate by withdrawing growth factors. Cellular differentiation of GCSC cells was confirmed from increased expression of known differentiation marker GFAP and diminished surface expression of known the GCSC marker CD133.

To explore quantitative changes in GBMSig expressions following differentiation, both proliferative and differentiating GCSCs were analyzed by flow cytometry. While decreases in surface expression levels of MRC2 (similar to isotype), SLC16A3 (38.5%±4.12 SEM), CD97 (70.77%±4.52 SEM) and HMOX1 (23.5%±1.92 SEM) in differentiating conditions relative to proliferating conditions were observed, enhanced expression of CD47 (21.43%±1.37 SEM) was observed in differentiating conditions relative to proliferation. There was no significant change in level for SLC16A1 expression during differentiation as measured by flow cytometry, but with fluorescent microscopy, a small population of GCSC cells were detected that were stained positive for SLC16A1 in proliferative conditions and diminished following differentiation.

Increase in expression of HMOX1—another putative GCSC marker in proliferative conditions and subsequent decrease in differentiating condition as observed by fluorescent microscopy, is also in good agreement with the flow cytometry data.

Thus the discrepancy that arose from flow cytometry analysis of SLC16A1 positive cells may be related to the averaging of SLC16A1 signal in flow cytometry due to rarity of the SLC16A1-positive cells among SN143-derived GCSC populations. In essence, the results demonstrated association of MRC2, SLC16A3, SLC16A1, HMOX1, and CD97 with proliferating primary GBM cells enriched in GCSC populations.

### Example 6

### Tumor Stratification

Brain tissues and blood from 4 male patients (SN132, SN143, SN154, and SN186) who underwent surgery at Swedish Hospital in Seattle, Wash. was used. Appropriate consents were received before the surgical procedure and specimen collection. Brain tissues of tumor and non-tumor origin were homogenized, enzymatically digested, clarified using C18, and spiked with synthetic C-terminally labeled (¹³C¹⁵N K/R) peptides for SRM analysis. As described above, d-SRM assays were developed by determining the retention time of each surrogate peptide in presence of corresponding tissue or serum isolates in prior runs, thus reducing the peptide retention time window during SRM run and improving the confidence in peptide identification across multiple isolates as could be evident from high correlation (R²>0.99) of peptide retention time among different isolates. In addition, analysis of multiple transitions (Q1-Q3) for individual peptides improved the precision and quality of SRM analysis.

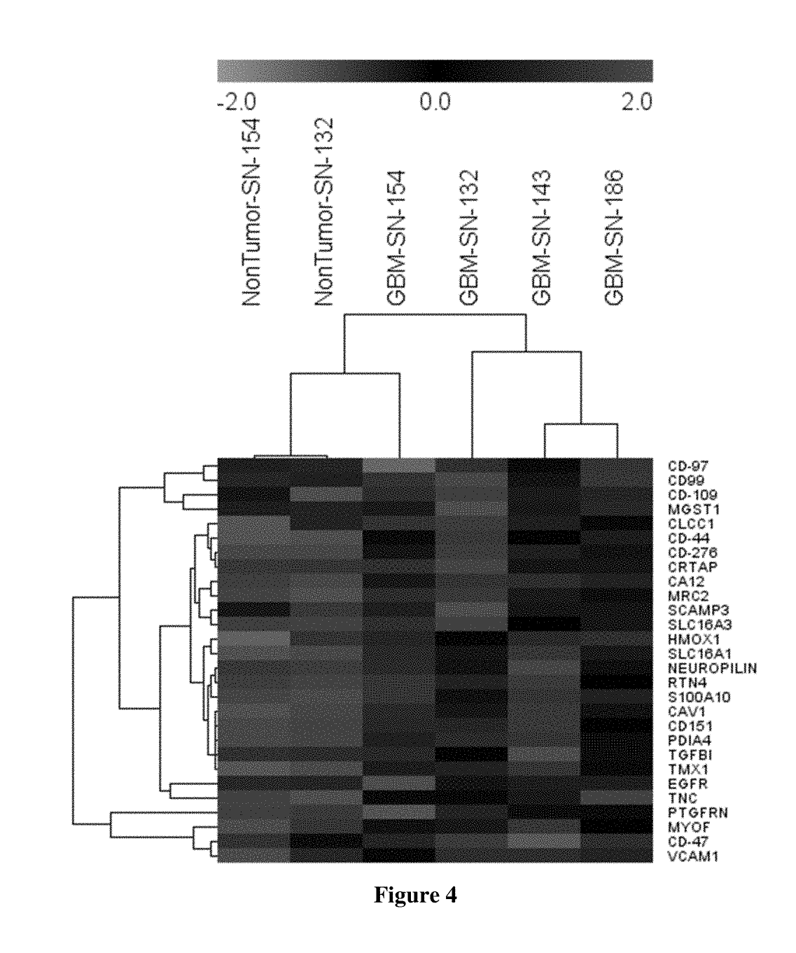

Relative expression of 21 of 33 GBMSig proteins was quantified in all four GBM tissues. Because of the rarity of non-tumor brain tissues, GBMSig expression in the four GBM tissues could only be compared with that in two of the non-tumor tissues that came from SN132 and SN154 subjects. After Z score transformation of SRM trace (ratio of endogenous to surrogate peptide) across six brain tissues (4 GBM and 2 non-tumors), 12 GBMSig proteins overexpressed in all four GBM tissues were observed in comparison to both the non-tumor tissues, represented as a heat-map as shown in FIG. 4.

Although majority of GBMSig proteins revealed differential expression between tumor and non-tumor regions of the brain, intratumor heterogeneities in GBMSig expression were clearly evident. To explore the level of heterogeneities among these four GBM patients, these tumors were stratified by employing qRT-PCR for 33 known gene panels as described by Phillips, et al.[2]. Patient SN154 was stratified as proliferative, SN186 as proneuronal, SN143 as mesenchymal and SN132 as intermediate. Each GBMSig protein (N=28) was ranked based on Z score across the four GBM tissues, resulting in categorizing 28 GBMSig into three groups. In comparison to GBMSig-transcriptome based stratification of TCGA, similar subtype-specific expression for 8 GBMSig proteins was observed.

CAV1, TGFBI, and CA12 were found relatively overexpressed in gliosarcoma SN143 similar to the mesenchymal-subtypes underlined in TCGA datasets. Similarly, in classical/proliferative SN154, relative overexpression of EGFR and reduced expression of S100A10 and NRP1 or in case of proneuronal SN186, relative overexpression of SLC16A3 and SCAMP3 at both the transcriptome and proteome levels was observed. In intermediate subtype SN132, expression patterns of both mesenchymal as evident from the overexpression of CAV1, TGFBI, and CA12 proteins, and proliferative as evident from the overexpression of EGFR were clearly visible. The expression pattern of selected GBMSig proteins is thus reminiscent of GBM heterogeneities at both transcriptome and proteome levels so as to enable GBM stratification.

### Example 7

### Expressions of Certain GBMSig Proteins after Tumor Resection

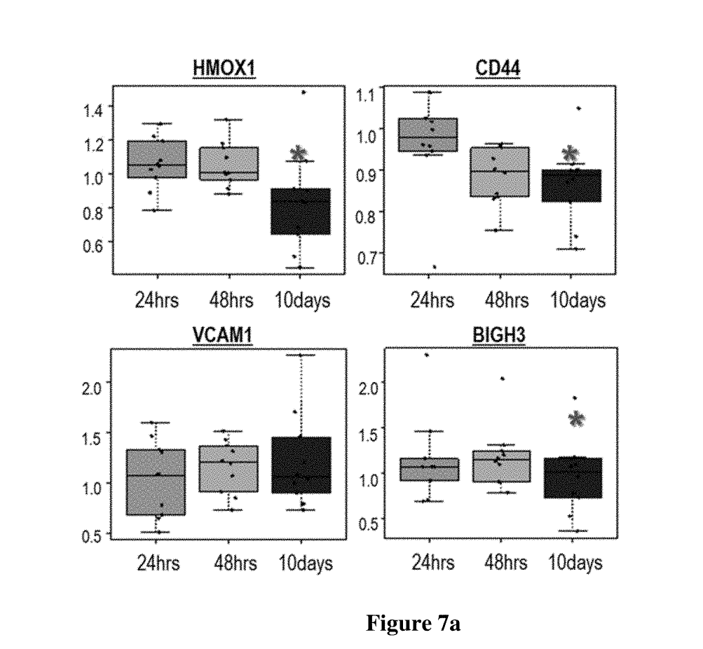

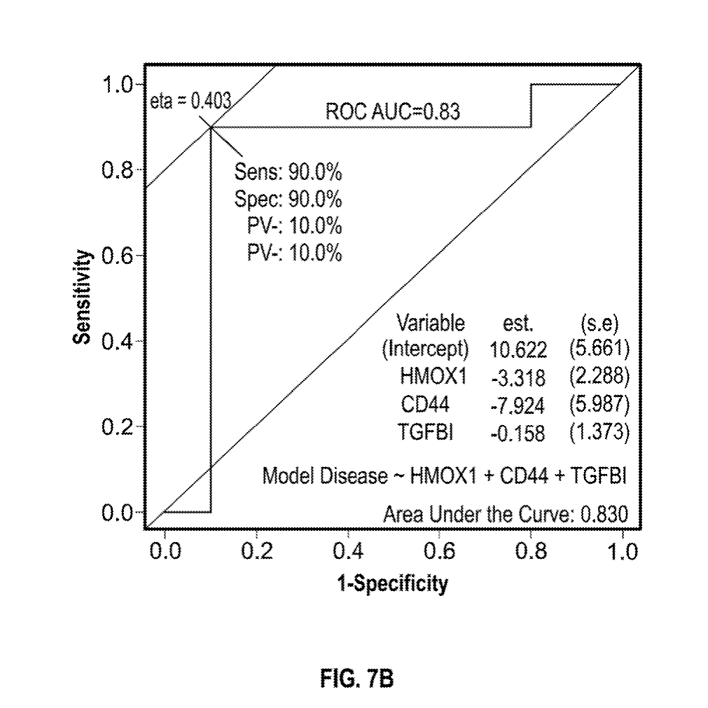

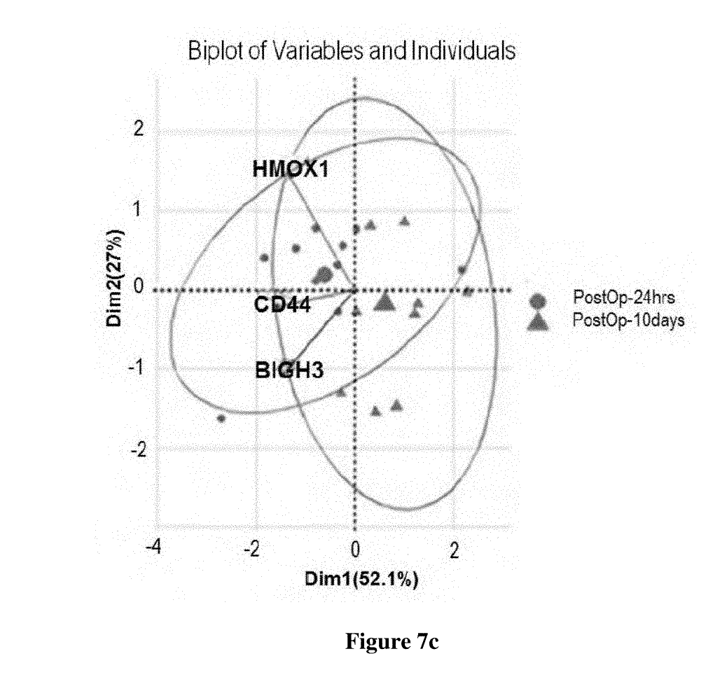

To demonstrate potential clinical utility in assaying changes of GBMSig proteins in the blood concentrations, changes in blood concentrations of HMOX1, CD44, VCAM1, and TGFBI (BIGH3) for ten GBM patients prior to and after tumor resection were examined. Blood samples were collected preoperatively and postoperatively at 24 hrs, 48 hrs, and ˜10 days postsurgery (first post-operative visit). From ELISA analysis, significant changes (p<0.05; ROC AUC of 0.83) in the blood concentrations of HMOX1, CD44, and TGFBI were observed. These results are shown in FIGS. 7a and 7b.

As shown in FIG. 7c, PCA analysis also revealed robust separation of 52.1% on PC1 and 27% on PC2 for changes in the blood concentrations of HMOX1, CD44, and TGFBI between early postoperative (24 hrs) and late postoperative (˜10 days) conditions. Together, the results may reflect treatment associated changes as demonstrated through expressions of GBMSig proteins.

1. A method for assessing the probability that a human subject is afflicted with glioblastoma multiforme (GBM) which method comprises:
a) assessing the level of at least one protein selected from the group consisting of HMOX1, CD44, VCAM1, and TGFBI in the blood or fraction thereof of a test subject;
b) comparing the level of said at least one of said proteins to the level of said protein in the blood or fraction thereof of normal subjects;
wherein a decreased level of CD44 and/or an increased level of HMOX1 and/or a decreased level of VCAM1 and/or a decreased level of TGFBI in the test subject as compared to normal subjects indicates the probability that said test subject is afflicted with GBM.

2. The method of claim 1 wherein the levels of at least two of said proteins are assessed in the test subject and compared to normal subjects.

3. The method of claim 2 wherein said two proteins are CD44 and HMOX1.

4. The method of claim 1 wherein said assessing in part a) is by SRM mass spectrometry or by immunoassay.

5. A solid support to which is bound in an ordered array, a reagent for the detection of each of the proteins HMOX1, CD44, VCAM1 and TGFBI.

6. A method for assessing the probability that a test subject is afflicted with GBM which method comprises:
a) contacting a sample of blood or a fraction thereof of said test subject with the ordered array of claim 5;
b) assessing the amount of at least one of HMOX1, CD44, VCAM1 and TGFBI bound to the corresponding reagent in said array;
c) comparing said amount to the amount observed in a similar assay performed on blood or fraction thereof of normal subjects;
wherein a decreased level of CD44 and/or an increased level of HMOX1 and/or a decreased level of VCAM1 and/or an increased level of TGFBI in the test subject as compared to normal subjects indicates the probability that said test subject is afflicted with GBM.

7. A method to determine whether a subject afflicted with GBM will respond to treatment with an inhibitor of TGF-β1, which method comprises assessing the level of at least one protein selected from the group consisting of TGFBI, ITGA7, TNC, DDR2, MRC2, MGST1, CLCC1, PTGFRN, CRTAP, CD109 and SLC16A1 in the blood or fraction thereof or in GBM tissue of said subject and comparing said level to that in normal subjects wherein an enhanced level of said protein in said test subjects indicates susceptibility to treatment with an inhibitor of TGF-β1.

8. The method of claim 7 which further includes assessing the levels of at least one protein selected from the group consisting of CD47, MYOF, ABCA1, S100A10, CA12 and SLC16A3 in the blood of said test subject, wherein higher levels of at least one of said proteins indicates susceptibility to treatment with an inhibitor of TGF-β1.

9. A method to determine whether a subject afflicted with GBM will respond to treatment with an inhibitor of TGF-β1, which method comprises assessing the level of at least one protein selected from the group consisting of HMOX1, SLC16A1, CD47 and MRC2 in blood or fraction thereof or in GBM tissue from said subject and comparing said level to that in normal tissue wherein an enhanced level of said protein in said GBM tissue as compared to normal tissue indicates susceptibility to treatment with an inhibitor of TGF-β1.

10. A composition which comprises an active agent that decreases the level of expression or the concentration of a protein selected from the group consisting of HMOX1, SLC16A1, CD47, MRC2, TGFBI, ITGA7, TNC, DDR2, MRC2, MGST1, CLCC1, PTGFRN, CRTAP, CD109 and SLC16A1 in the blood or tissues of a subject for use in a method to treat GBM in said subject.

11. A method to classify GBM tissue, which method comprises assessing the level of at least one GBMSig protein in a tissue and comparing said level to that in normal tissue, wherein an enhanced level of ASPH, SCAMP3, CLCC1 and/or CADM1 in said tissue indicates the tissue is proneuronal; and enhanced level of CD44, TTG47 and/or EGFR in said tissue indicates the tissue is classical, and an increased level of CAV and/or TGFBI in said tissue indicates the tissue is mesenchymal.

12. A method for assessing the probability that a human subject is afflicted with glioblastoma multiforme (GBM) which method comprises assessing the level of at least one protein selected from the group consisting of the 33 proteins of GBMSig in the brain tissue, tumor cells, blood, or fraction thereof of a test subject and comparing the level of said protein to that of said protein in the brain tissue, tumor cells or blood, or fraction thereof of normal subjects, whereby a difference in the level in the test subject as compared to normal subjects indicates the probability that the test subject is afflicted with GBM,
wherein said 33 proteins are ABCA1, ASPH, CA12, CADM1, CAV1, CD109, CD151, CD276, CD44, CD47, CD97, CD99, CLCC1, CRTAP, DDR2, EGFR, HMOX1, ITGA7, MGST1, MRC2, MYOF, NRP1, PDIA4, PTGFRN, RTN4, S100A10, SCAMP3, SLC16A1, SLC16A3, TGFBI, TMX1, TNC and VCAM1.

13. The method of claim 12 wherein said at least one protein is selected from the group consisting of DDR2, PDIA4, CADM1, ITGA7, MRC2, MYOF, NRP1, RTN4, TNC, SCAMP3 and CD47.

14. A solid support to which is bound in an ordered array reagents for detection of at least three proteins selected from the group consisting of ABCA1, ASPH, CA12, CADM1, CAV1, CD109, CD151, CD276, CD44, CD47, CD97, CD99, CLCC1, CRTAP, DDR2, EGFR, HMOX1, ITGA7, MGST1, MRC2, MYOF, NRP1, PDIA4, PTGFRN, RTN4, S100A10, SCAMP3, SLC16A1, SLC16A3, TGFBI, TMX1, TNC and VCAM1.

15. A method for assessing the probability that a test subject is afflicted with GBM which method comprises:
a) contacting a sample of blood, brain tissue, tumor tissue or a fraction thereof of said test subject with the ordered array of claim 14;
b) assessing the amount of at least three of proteins that are ABCA1, ASPH, CA12, CADM1, CAV1, CD109, CD151, CD276, CD44, CD47, CD97, CD99, CLCC1, CRTAP, DDR2, EGFR, HMOX1, ITGA7, MGST1, MRC2, MYOF, NRP1, PDIA4, PTGFRN, RTN4, S100A10, SCAMP3, SLC16A1, SLC16A3, TGFBI, TMX1, TNC and VCAM1bound to the corresponding reagent in said array;
c) comparing said amounts to the amounts observed in a similar assay performed on blood, brain tissue, tumor tissue or fraction thereof of normal subjects;
whereby a difference in the levels said at least three proteins in the test subject as compared to normal subjects indicates the probability that the test subject is afflicted with GBM.
